(19) United States
(12) Patent Application Publication
(10) Pub. No.: US 2025/0001197 A1
(43) Pub. Date: Jan. 2, 2025

(54) MOBILE INFRARED AND NEGATIVE OXYGEN ION EXPOSURE CHAMBER

(71) Applicant: Henan Qianpeng Biopharmaceutical Co. Ltd., Zhengzhou (CN)

(72) Inventors: Hehui Sun, Zhengzhou (CN); Tianen Sun, Zhengzhou (CN); Tianyu Sun, Zhengzhou (CN); Xiaomei Wang, Zhengzhou (CN); Sentao Yang, Zhengzhou (CN)

(21) Appl. No.: 18/885,174

(22) Filed: Sep. 13, 2024

(30) Foreign Application Priority Data
Jun. 28, 2024 (CN) 202410856872.1

(51) Int. Cl. A61N 5/06; A61H 37/00

(52) U.S. Cl. CPC A61N 5/0613; A61H 37/00; A61H 2201/0149; A61H 2201/10; A61H 2203/0431; A61N 2005/0638; A61N 2005/0654; A61N 2005/0659

(57) ABSTRACT

A mobile infrared negative oxygen ion exposure chamber. The chamber includes a base plate assembly having four corners, four enclosure plates arranged about the base plate assembly to form an enclosure, four support columns, a top plate assembly, an infrared radiation generator, a negative oxygen ion generator, and two fixed wheels and one removeable swiveling wheel for moving the chamber.

### CROSS-REFERENCE TO RELATED APPLICATIONS

This application claims the benefit of Chinese Patent Application No. 202410856872.1, filed on Jun. 28, 2024. The entire content of the prior application is hereby incorporated by reference herein.

### BACKGROUND

Existing infrared negative oxygen ion chambers are designed to provide an inviting recuperation space; to purify the air inside by removing harmful airborne particles such as smoke, dust, and bacteria gases; and to improve physiological functions of the individual using the chamber.

Infrared rays and negative oxygen ions are thought to regulate human physiological functions, eliminate fatigue, improve sleep, prevent respiratory diseases, and cardiovascular and cerebrovascular diseases, and also have a positive effect on lowering blood pressure, increasing appetite, and enhancing skin elasticity.

Far-infrared radiation (“FIR”) is an electromagnetic wave with a wavelength between 2.5 μm to 1000 μm. FIR has strong penetrating ability and can penetrate into the human body. FIR is believed to have anti-inflammatory and analgesic properties, especially for superficial pain. It can also promote blood circulation, improve immunity, and help remove accumulated waste from the body.

Negative oxygen ions are negatively charged oxygen molecules. They can actively capture positively charged particles in the air, such as dust and pollutants, and cause them to condense and precipitate, thereby effectively removing particulate pollutants from the air.

Typical negative oxygen ion rest chambers are designed to solve the need for healthy short rest in multiple scenarios, such as airports, shopping malls, office buildings, outdoor squares and parks and other public places. These chambers are often used by hospital attendants, airline crews, and airport control tower personnel to take short breaks from these stressful occupations.

However, existing chambers still have some common shortcomings as follows.

Due to the wear and tear of the chamber during use, the chamber may be damaged, and regular inspection and maintenance are required to ensure safety. However, as there is no standard chamber, their unique structures make it difficult to replace and maintain the structural components of the chamber.

Second, during use, due to poor light transmittance, individuals undergoing oxygen therapy may need to be accompanied by outsiders to prevent accidental coma in the oxygen chamber.

Third, the manufacturing and maintenance costs of negative oxygen ion chambers are relatively high, which may not be affordable by all users.

There is a need for chamber structures suitable for consumers that lack the shortcomings set out above.

### SUMMARY

To satisfy the need set out above, a mobile infrared and negative oxygen ion exposure chamber is provided. The chamber includes a base plate assembly having four corners, four enclosure plates arranged about the base plate assembly to form an enclosure, four support columns, a top plate assembly, means for generating infrared radiation (IR), and means for generating negative oxygen ions.

One of the enclosure plates has an opening for entering the enclosure. The wall sections are reversibly joined at the support columns, which are in turn each attached to one corner of the base. The top plate is in contact with the top edge of each of the enclosure plates and the top of each of the support columns. The base plate assembly includes two fixed wheels and one removeable swiveling wheel configured for moving the chamber in a desired direction, and the chamber is configured to operate at a concentration of at least 5,000 (e.g., at least 20,000, at least 10,000, at least 8,000, 5000-20,000, and 6,000-15,000) negative oxygen ions per cubic meter of internal volume of the enclosure.

The advantages of the disclosed chamber are (i) the chamber structure does not require specially customized materials, thereby keeping the cost of the chamber lower than typical exposure chambers and also keeping future maintenance costs low, (ii) the chamber is easy to assemble and to disassemble for parts and equipment replacement, (iii) the internal structure of the chamber is flexible, providing seating and lying positions, (iv) the wall section opening can be transparent to admit natural light, and (v) vents and mechanical push-pulls can be included that can be opened manually in an emergency.

The details of one or more embodiments are set forth in the description and the examples below. Other features, objects, and advantages will be apparent from the detailed description, from the drawings, and also from the appended claims.

### BRIEF DESCRIPTION OF THE DRAWINGS

The description below refers to the accompanying drawings, of which:

FIG. 1 is a schematic diagram of the overall structure of an embodiment of a mobile infrared and negative oxygen ion exposure chamber.

FIG. 2 is a cross-sectional view of one embodiment of an enclosure plate showing a multi-layer structure.

FIG. 3 is a view of the underside of the base plate assembly.

FIG. 4 is a top-down view of the base plate assembly.

FIG. 5 shows a view of the underside of the top plate assembly.

FIG. 6 shows a top-down view of the top plate assembly.

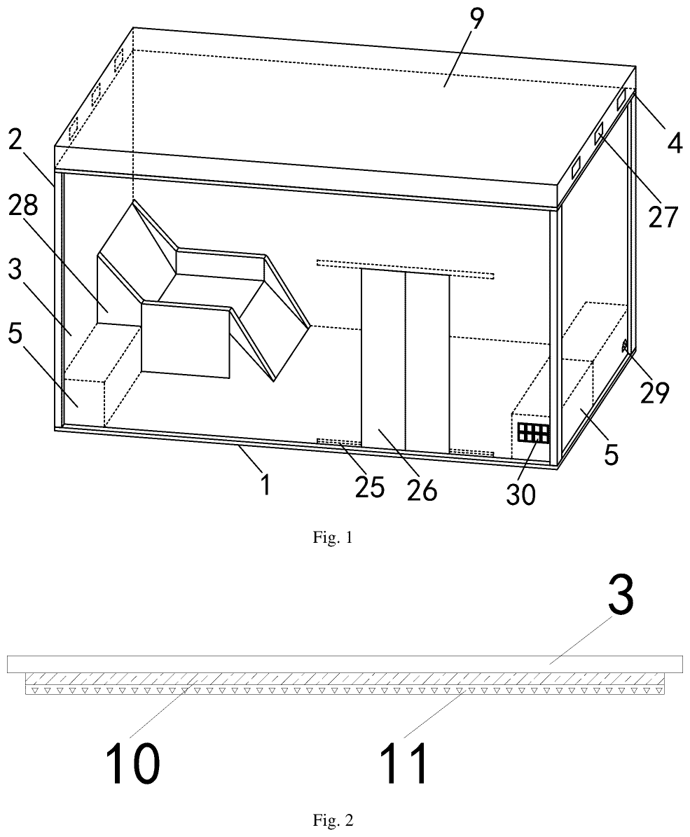

FIGS. 1-6 show the following components: base plate assembly (1), support column (2), enclosure plate (3), top plate assembly (4), cabinet assembly (5), fixed wheel (6), infrared lamp (7), annular anion-emitting needle assembly (8), photovoltaic panel (9), electric heating layer (10), solid anion layer (11), support beam (12), base support panel (13), reinforcement beam (14), bottom support column hole (15), removable swiveling wheel (16), legs (17), enclosure plate slot (18), top support beam (19), top support panel (20), top support column hole (21), infrared lamp mount (22), needle emitter array mounting plate (23), top plate assembly air guide (24), motorized sliding rails (25), tempered glass door (26), air duct opening (27), massage chair (28), power port (29), switch panel (30), swiveling wheel mounting holes (31), base support panel mounting slot (32), and top support panel mounting slot (33).

### DETAILED DESCRIPTION

As summarized above, a mobile infrared and negative oxygen ion exposure chamber is disclosed in which the chamber includes a base plate assembly having four support columns, each mounted at a corner of the base plate assembly, four enclosure plates arranged about the base plate assembly to form an enclosure, a top plate assembly attached to the top of the support columns and the tops of the enclosure plates, means for generating infrared radiation (IR), and means for generating negative oxygen ions.

The enclosure plates can be made of any suitable material, such as wood, (e.g., bamboo), plastic, or steel. In a preferred embodiment, the enclosure plates are made of wood.

The base plate assembly includes a base support panel that can be formed of wood, steel, concrete, or concrete covered with applied wood boards.

In a specific chamber, at least one of the enclosure plates has multiple functional layers attached thereto. For example, the enclosure plate can have attached to it an electric heating layer and a solid anion layer arranged in order such that the solid anion layer faces the inside of the enclosure and the electric heating layer is sandwiched between the solid anion layer and the enclosure plate.

The solid anion layer can be formed of a material that spontaneously or inductively produces negative oxygen ions. For example, the layer can be formed of tourmaline. In particular embodiments, activation of the electric heating layer induces release of anions from the solid anion layer.

As mentioned in the SUMMARY section, the chamber includes means for generating negative oxygen ions. The means for generating negative oxygen ions can be an annular anion-emitting needle assembly that is controlled by an anion emission controller electrically connected to it. The annular anion-emitting needle assembly can be located, for example, on the underside of the top plate assembly of the chamber.

The annular anion-emitting needle assembly can operate as follows. (1) Electrical processing: The generator circuit first processes input DC or AC power through an electromagnetic interference (EMI) processing circuit and overload protection circuit to ensure the stability and safety of the current. (2) High voltage conversion: The processed current is amplified to high AC voltage through pulse circuits and overvoltage current limiting circuits. (3) Rectification and filtering: The high-voltage AC current is rectified and filtered to obtain pure DC negative high voltage. (4) Release negative ions: Finally, these negative high voltages are applied to the anion-emitting needles, causing air molecules to gain extra electrons, thereby releasing negative ions into the interior of the chamber.

Additional means for generating negative oxygen ions can be the solid anion layer attached to one or more of the enclosure plates. In a particular chamber, the means for generating negative oxygen ions is a combination of the annular anion-emitting needle assembly and the solid anion layer.

The means for generating negative oxygen ions is capable of providing a concentration of at least 10,000 negative oxygen ions per cubic meter of internal volume of the enclosure.

To repeat from above, the mobile infrared and negative oxygen ion exposure chamber includes means for generating IR, which can be far-infrared (FIR). As defined herein, FIR is an electromagnetic wave with a wavelength ranging from 2.5 μm to 1000 μm. A FIR generator is a device that works on the principle of electromagnetic wave radiation. It employs special materials and excites them to emit far-infrared rays.

The means for generating FIR can be, but is not limited to, a material that emits FIR spontaneously or a material that emits FIR in response to an electric current, e.g., resistance wire and carbon fibers. In one example, the means for generating FIR is an infrared lamp mounted on the underside of the top plate. In another example, the FIR is emitted by the electric heating layer that is part of at least one enclosure plate. In this embodiment, the electric heating layer is a FIR electric heating plate.

In a specific example of the mobile infrared and negative oxygen ion exposure chamber, the discharge needles are arranged concentrically around the infrared lamp on the underside of the top plate.

For ease of construction and maintenance, the mobile infrared and negative oxygen ion exposure chamber includes one or more cabinet assemblies reversibly fixed to the base plate assembly. The cabinet assemblies provide a location for electronics that control the chamber or that provide diagnostic information for maintaining the chamber in good operating order.

One cabinet assembly is used to house the anion emission controller. This cabinet assembly can be removably fixed to the base plate assembly along one of the enclosure plates. The cabinet assembly includes an access panel and can also be easily removed to either repair or replace the anion emission controller. In a particular chamber, the cabinet assembly housing the anion emission controller can serve as a bench for seating within the chamber. In a preferred embodiment, a separate massage chair is provided inside the chamber.

The cabinet assembly described in the preceding paragraph can also house an infrared control unit. In an embodiment, the infrared control unit is housed in another cabinet assembly also reversibly fixed to the base plate assembly.

Another cabinet assembly can include detection equipment. The detection equipment serves two purposes. During use of the chamber, the detection equipment is used to monitor and adjust parameters within the enclosure, including, but not limited to, temperature, humidity, FIR intensity, and negative oxygen ion concentration. When these parameters fall outside of pre-set ranges, the detection equipment can signal to control circuitry to bring the parameters back within range. In one example, if the negative oxygen ion concentration falls below a pre-set level, e.g., 10,000 ions/m³, the detection equipment sends a signal to the to the anion emission controller that in turn regulates the annular anion-emitting needle assembly to increase production of negative oxygen ions.

The detection equipment can also be used in a diagnostic mode during servicing of the mobile infrared and negative oxygen ion exposure chamber. Service personnel can attach diagnostic equipment to the detection equipment through an external switch panel to monitor for proper operation of the chamber and to diagnose any operational problems.

The mobile infrared and negative oxygen ion exposure chamber can also include a power supply cabinet (reversibly fixed to the base) that houses a power supply that provides stable current and voltages to all of the internal circuitry. In an exemplary chamber, an opening is present in the enclosure plate adjacent to the power supply cabinet such that a power cord can be attached to the power supply from the outside of the chamber to provide electrical power. In a different exemplary chamber, one or more solar panels are mounted on the outer surface of the top plate to supply electricity to the power supply.

It should be noted that the number of cabinets is not fixed, as certain cabinets described above can be combined into a single cabinet. For example, the power supply cabinet can be combined with the cabinet housing the detection equipment.

When constructing the mobile infrared and negative oxygen ion exposure chamber, it is important to choose the appropriate type and output level of the annular anion-emitting needle assembly according to the interior volume of the chamber. It is also important to consider the positive ion balance. Although negative ions are beneficial to the human body, too many positive ions or negative ions may cause adverse effects, so a proper ion balance should be maintained.

The mobile infrared and negative oxygen ion exposure chamber set forth above has certain advantages over existing chambers as follows: (i) the base plate assembly and the top plate assembly, as well as the support columns and the enclosure plates, can be conveniently assembled or replaced in situ (ii) the fixed wheels can be adaptively adjusted to the position of the chamber after assembly, (iii) the annular, i.e., ring-shaped, anion needle array is set in the upper part of the chamber to uniformly release anions and maintain the concentration inside the chamber, and (iv) the heat released from the electric heating layer heats up the chamber and also further promotes the release of anion from the solid anion layer.

The interconnections between the base plate assembly, support columns, enclosure plates, and top plate assembly provide support and stiffness to the chamber, while also being convenient for assembly and disassembly. The removable swiveling wheel permits convenient control of the moving direction and supports the chamber when it is being moved. Once the chamber is at the desired location, the swiveling wheel can be removed and the chamber can rest stably on the legs and fixed wheels.

Without further elaboration, it is believed that one skilled in the art can, based on the disclosure herein, utilize the present disclosure to its fullest extent. The following specific examples are, therefore, to be construed as merely descriptive, and not limitative of the remainder of the disclosure in any way whatsoever.

### Example 1

An embodiment of a mobile infrared and negative oxygen ion exposure chamber is depicted in FIGS. 1-6. As shown in FIG. 1, the chamber includes a base plate assembly 1, with support columns 2 provided at the corners of the base plate assembly 1. Enclosure plates 3 are provided between adjacent support columns 2 to form an enclosure, and a top plate assembly 4 is provided on top of the enclosure to form the chamber.

FIG. 2 provides details of the enclosure plate 3. At least one enclosure plate has a layered structure in which an electric heating layer 10 is provided on an inner surface of at least one of the enclosure plates 3 and a solid anion layer 11 is provided on an inner surface of the electrically heated layer 10 such that the solid anion layer 11 is exposed to the interior of the chamber once assembled. The solid anion layer 11 can be a composite plate containing tourmaline, and the electric heating layer 10 can be an infrared electric heating plate or an electric heating wire,. In an embodiment, the electric heating layer 10 is a far-infrared electric heating plate.

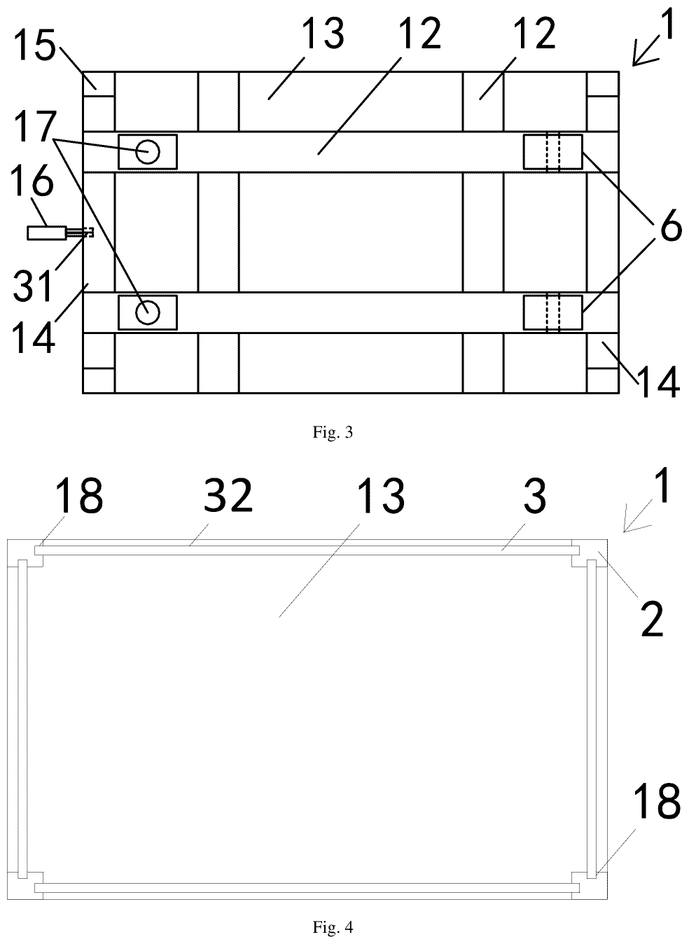

The underside of the base plate assembly 1 is shown in FIG. 3. It includes base support panel 13 stiffened by four support beams 12, two of which are in a transverse direction, and, along two opposite ends of the support panel 13, two reinforcement beams 14. Each corner of the base plate assembly 1 includes a bottom support column hole 15 that extends through the support panel 13 into the reinforcement beam 14. Each bottom support column hole 15 is for receiving the support column 2. Each transversely oriented support beam 12 includes a fixed wheel 6 proximate to one end and a leg 17 proximate to the other end. The legs 17 provide stability for the chamber when in use, and the fixed wheels 6 provide mobility for the chamber in conjunction with a removable swiveling wheel 16 that is reversibly attached to one of the reinforcement beams 14 with fasteners placed into the swiveling wheel mounting holes 31.

FIG. 4 shows a top view of the base plate assembly 1 of a partially assembled chamber. Each support column 2 includes two enclosure plate slots 18 extending along its length. The enclosure plate slots 18 receive the vertical edges of the enclosure plates 3. The bottom horizontal edge of each enclosure plate 3 is inserted into a base support mounting slot 32 that extends around the perimeter of the base support panel 13.

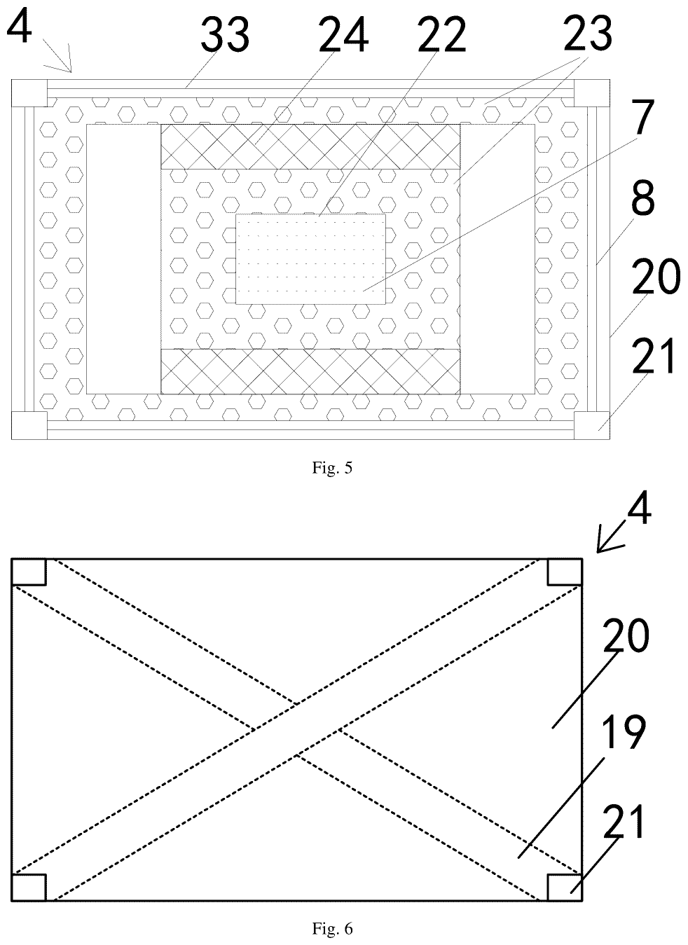

FIG. 5 shows the top plate assembly 4 as viewed from the interior of the chamber. Mounted on the top support panel 20 is an infrared lamp 7 attached to an infrared lamp mount 22. Surrounding the infrared lamp 7 is an annular anion-emitting needle assembly 8 that includes one or more needle emitter array mounting plates 23. Provided between the needle emitter array mounting plates 23 and the infrared lamp mount 22 is a top plate assembly air guide 24. Further, the top plate assembly 4 includes a top support panel 20 having around its periphery a top support panel mounting slot 33 for receiving the upper edges of each enclosure plate 3 when the chamber is assembled. Four top support column holes 21 are also included to receive the upper ends of the support columns 2.

Shown in FIG. 6 are two crossed top support beams 19 that are provided for stiffening the top plate 20 of the top plate assembly 4. The top support beams 19 are located between the top plate 20 and a photovoltaic panel 9 provided on the outer upper surface of the chamber (see FIG. 1). The photovoltaic panel 9 is included to provide supplementary power when the chamber is installed outdoors.

The top plate assembly 4 also includes at least one air duct opening 27 between the top plate 20 and a photovoltaic panel 9. The air duct opening 27 is connected to an air duct and blower to ventilate the cabinet.

Additional features of the mobile infrared and negative oxygen ion exposure chamber are shown in FIGS. 1-6. One of the enclosure plates 3 includes an entrance, e.g., a tempered glass door 26, to provide access to the chamber. The tempered glass door 26 is mounted on motorized rail 25 to facilitate opening and closing. The photovoltaic panel 9 is electrically connected to the electric heating layer 10 via control circuitry. In the embodiment shown in FIG. 1, a massage chair 28 is provided inside the chamber.

To control the functions of the mobile infrared and negative oxygen ion exposure chamber, at least one cabinet assembly 5 is mounted inside the chamber. In the embodiment of the chamber shown in FIG. 1, two cabinet assemblies 5 are provided that are arranged on different sides of the chamber. The two cabinet assemblies contain, respectively, (i) an anion emission controller and anion detection unit and (ii) an infrared control unit and a power supply interface. The anion emission controller is electrically connected to the annular anion-emitting needle assembly 8 and the infrared control unit is electrically connected to the infrared lamp 7. A power supply cabinet can also be provided that includes a power port 29 that accessible from the exterior of the chamber. The power port can accept an electrical cable from an external power source. Finally, a switch panel 30 is installed in one of the cabinet assemblies for operating the chamber and for diagnostic purposes.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A mobile infrared and negative oxygen ion exposure chamber, the chamber comprising:
a base plate assembly having four corners;
four enclosure plates arranged about the base plate assembly to form an enclosure, one of the enclosure plates having an opening for entering the enclosure;
four support columns each having a top end, a bottom end and four surfaces extending therebetween, two adjacent surfaces of which being reversibly joined to two enclosure plates and the bottom end of each support column being reversibly attached to a single one of the four corners of the base plate assembly;
a top plate assembly in contact with a top edge of each of the enclosure plates and the top end of each of the support columns;
means for generating infrared radiation (IR); and
means for generating negative oxygen ions,
wherein the base plate assembly includes two fixed wheels and one removeable swiveling wheel configured for moving the chamber in a desired direction, and the chamber is configured to operate at a concentration of at least 10,000 negative oxygen ions per cubic meter of internal volume of the enclosure.

2. The chamber of claim 1, wherein at least one of the enclosure plates includes on an inner surface thereof an electric heating layer and a solid anion layer configured such that the solid anion layer covers the electric heating layer and is exposed to the interior of the chamber.

3. The chamber of claim 1, wherein the means for generating negative oxygen ions includes an annular anion-emitting needle assembly attached to an interior side of the top plate assembly and an anion emission controller electrically connected to the annular anion-emitting needle assembly.

4. The chamber of claim 3, further comprising a first cabinet assembly removably fixed to the base plate assembly along one of the enclosure plates, wherein the anion emission controller is housed within the first cabinet assembly.

5. The chamber of claim 4, wherein the means for generating IR comprises an infrared lamp arranged on the underside of the top plate assembly.

6. The chamber of claim 5, wherein the annular anion-emitting needle assembly is arranged concentrically around the infrared lamp.

7. The chamber of claim 5, further comprising a second cabinet assembly housing an infrared control unit that is electrically connected to the infrared lamp.

8. The chamber of claim 2, further comprising one or more photovoltaic panels for supplying electricity to the electric heating layer, wherein the one or more photovoltaic panels are mounted on an outer surface of the top plate assembly or mounted on an outer surface of the enclosure plates.

9. The chamber of claim 2, wherein the solid anion layer comprises tourmaline and the electric heating layer is a far-infrared heating plate.

10. The chamber of claim 5, further comprising a third cabinet assembly that houses a power supply interface and an anion detection unit.

11. The chamber of claim 1, further comprising two legs attached to an underside of the base plate assembly to reduce movement of the chamber after installation.

12. The chamber of claim 6, further comprising an air guide located between the annular anion-emitting needle assembly and the infrared lamp, the air guide for evenly distributing negative ions generated by the annular anion-emitting needle assembly and heat generated by the infrared lamp.

13. The chamber of claim 1, wherein the top plate assembly includes one or more air duct openings connected to an air duct and blower for regulating temperature inside the chamber.

14. The chamber of claim 1, wherein the opening for entering the chamber comprises one or more tempered glass doors mounted on a motorized rail.

15. The chamber of claim 1, further comprising a massage chair mounted on the base plate assembly in the interior of the chamber.
